(19) United States
(12) Patent Application Publication
(10) Pub. No.: US 2026/0265724 A1
(43) Pub. Date: Sep. 10, 2026

(54) BIOSENSOR COMPRISING A MODIFIED BETA-LACTAMASE AND USES THEREOF

(71) Applicant: THE HONG KONG POLYTECHNIC UNIVERSITY, Hong Kong (CN)

(72) Inventors: Yun-Chung LEUNG, Hong Kong (CN); Kwok-Yin WONG, Hong Kong (CN); Sai-Fung CHUNG, Hong Kong (CN)

(21) Appl. No.: 18/993,653

(22) PCT Filed: Jul. 12, 2023
(86) PCT No.: PCT/CN2023/106980

§ 371 Date: Jan. 13, 2025

Related U.S. Application Data
(60) Provisional application No. 63/368,373, filed on Jul. 14, 2022.

(51) Int. Cl. C12N 9/86; C12Q 1/34; G01N 33/58

(52) U.S. Cl. CPC C12N 9/86; C12Q 1/34; C12Y 305/02006; G01N 33/582; G01N 2333/986

(57) ABSTRACT

The present invention provides a protein including a polypeptide having at least 95% sequence homology with an amino acid sequence selected from a group consisting of SEQ ID NOs: 1 to 5, where the amino acid at position 70 of SEQ ID NOs: 1-5 must be cysteine. The present invention also provides a biosensor including the present protein. The present invention further provides methods of using the present biosensor for identifying a beta-lactam and a beta-lactamase inhibitor.

### CROSS-REFERENCE TO RELATED APPLICATION

This application claims priority from PCT international patent application number PCT/CN2023/106980, filed on Jul. 12, 2023, which claims priority from U.S. provisional patent application Ser. No. 63/368,373 filed Jul. 14, 2022, the disclosure of which are incorporated herein by reference in their entirety.

### REFERENCE TO SEQUENCE DISCLOSURE

The sequence listing file under the file name “P24506PCT00_sequence_listing.xml” submitted in ST.26 XML file format with a file size of 11 KB created on Jul. 7, 2023 is incorporated herein by reference.

### TECHNICAL FIELD

The present invention relates to a biosensor comprising a modified beta-lactamase, in particular, a biosensor comprising a fluorescent labelled metallo-beta-lactamase (MBL). The present invention also relates to uses of the present biosensor in various applications including detection and screening of potential drugs for treating carbapenem-resistant bacterial infections.

### BACKGROUND

The following list of references are cited hereinafter:
1. Au H-W, Tsang M-W, Chen Y W, So P-K, Wong K-Y, Leung Y-C (2020) BADAN-conjugated β-lactamases as biosensors for β-lactam antibiotic detection. PLOS ONE 15(10): e0241594;
2. Brem, J.; van Berkel, S. S.; Zollman, D.; Lee, S. Y.; Gileadi, O.; McHugh, P. J.; Walsh, T. R.; McDonough, M. A.; Schofield, C. J., Structural Basis of Metallo-beta-Lactamase Inhibition by Captopril Stereoisomers. Antimicrob Agents Chemother 2016, 60 (1), 142-50;
3. Chan, P. H.; So, P. K.; Ma, D. L.; Zhao, Y.; Lai, T. S.; Chung, W. H.; Chan, K. C.; Yiu, K. F.; Chan, H. W.; Siu, F. M.; Tsang, C. W.; Leung, Y. C.; Wong, K. Y., Fluorophore-Labeled beta-Lactamase as a Biosensor for beta-Lactam Antibiotics: a Study of the Biosensing Process. J Am Chem Soc 2008, 130 (20), 6351-61;
4. Khan, A. U., Maryam, L. & Zarrilli, R. Structure, Genetics and Worldwide Spread of New Delhi Metallo-β-lactamase (NDM): a threat to public health. BMC Microbiol 17, 101 (2017);
5. Klingler, F. M.; Wichelhaus, T. A.; Frank, D.; Cuesta-Bernal, J.; El-Delik, J.; Muller, H. F.; Sjuts, H.; Gottig, S.; Koenigs, A.; Pos, K. M.; Pogoryelov, D.; Proschak, E., Approved Drugs Containing Thiols as Inhibitors of Metallo-beta-lactamases: Strategy To Combat Multidrug-Resistant Bacteria. J Med Chem 2015, 58 (8), 3626-30;
6. Liang Z, Li L, Wang Y, Chen L, Kong X, et al. (2011) Molecular Basis of NDM-1, a New Antibiotic Resistance Determinant. PLoS ONE 6(8): e23606;
7. Ma, B.; Fang, C.; Lu, L.; Wang, M.; Xue, X.; Zhou, Y.; Li, M.; Hu, Y.; Luo, X.; Hou, Z., The Antimicrobial Peptide Thanatin Disrupts the Bacterial Outer Membrane and Inactivates the NDM-1 Metallo-beta-Lactamase. Nat Commun 2019, 10 (1), 3517;
8. Matsumoto, S., Singley, C. M., Hoover, J., Nakamura, R., Echols, R., Rittenhouse, S., et al. (2017). Efficacy of cefiderocol against carbapenem-resistant gram-negative bacilli in immunocompetent-rat respiratory tract infection models recreating human plasma pharmacokinetics. Antimicrob. Agents Chemother. 61, e00700-17;
9. Shirley, M. (2018). Ceftazidime-avibactam: a review in the treatment of serious gram-negative bacterial infections. Drugs 78, 675-692.
10. Tsang M W, Chan P H, So P K, Ma D L, Tsang C W, Wong K Y, Leung Y C. Engineered Amp C B-lactamase as a fluorescent screening tool for class C B-lactamase inhibitors. Anal Chem. 2011 Mar. 15; 83(6):1996-2004.
11. Wang X, Lu M, Shi Y, Ou Y, Cheng X (2015) Discovery of Novel New Delhi Metallo-β-Lactamases-1 Inhibitors by Multistep Virtual Screening. PLoS ONE 10(3): e0118290;
12. Zhanel, G. G., Golden, A. R., Zelenitsky, S., Wiebe, K., Lawrence, C. K., Adam, H. J., et al. (2019). Cefiderocol: a siderophore cephalosporin with activity against carbapenem-resistant and multidrug-resistant gram-negative bacilli. Drugs 79, 271-289.

Beta-lactams (β-lactams) such as penicillin, carbapenems, cephalosporins and monobactams are commonly used antibiotics in modern medicine for treating Gram negative bacterial infections. Overuse or abuse of β-lactams is a root cause of increasing emergence of antibiotic-resistant bacteria which are capable of producing beta-lactamases (β-lactamases), a class of enzymes that hydrolyze β-lactams (FIG. 1), can mediate drug resistance to beta-lactam antibiotics. β-lactamases can be classified into four categories, namely Ambler classes A, B, C and D, where classes A, C and D beta-lactams contain active-site serine beta-lactamase (SBLs), while class B beta-lactams contain zinc-dependent metallo-beta-lactamases (MBLs). Acquisition of beta-lactamases, especially MBLs, by bacteria contributes largely to emergence of carbapenem-resistant bacteria, thereby possibly increasing patients' morbidity and mortality, and medical burdens. One of the most common MBLs, the New Delhi Metallo-beta-lactamase 1 (NDM-1), has raised awareness in the past 14 years as its ability to bind and hydrolyze most beta-lactam antibiotics make the corresponding Gram-negative bacteria resistant to a broad range of beta-lactam antibiotics. The carbapenem-resistant bacteria including carbapenem-resistant Enterobacterales (CRE), carbapenem-resistant Pseudomonas aeruginosa, and carbapenem-resistant Acinetobacter baumannii (A. baumannii) have already been listed by the WHO as the most emergent drug-resistant pathogens that are urgently needed for new antibiotics. NDM-1 are prone to gene mutations which result in NDM-type β-lactamases variants with different substrates and kinetic profiles. At least seventeen variants of NDM-type gene differing in one or two residues of amino acids at distinct positions have been reported so far among different species of bacteria from different countries. Bacteria acquiring NDM-type β-lactamases are usually regarded as “superbugs” because only limited antibiotics are available to treat conditions caused by these bacteria. However, none of clinically effective inhibitor is available for NDM-1-containing bacteria. To this end, effective drug screening platform based on NDM-type β-lactamases must be available for discovery new drug candidates and antibiotic/inhibitor. NDM-1 is regarded as the ancestor of members in the NDM family and shares a common tertiary structure to other NDM-type β-lactamases.

Inactivation or inhibition of NDM-1 appears to be a promising target to resensitize carbapenem-resistant bacteria to a majority of existing β-lactam antibiotics, especially carbapenem compounds, such as carbapenem antibiotic meropenem. From a binding mechanism point of view, a cysteine residue at the 208th position which is buried within an active binding site towards most β-lactams is responsible for binding to Zn²⁺ to catalyze hydrolysis of β-lactams. Shirley (2018) provided ceftazidime-avibactam (CAZ-AVI), newly approved combinations of beta-lactam/beta-lactamase inhibitors, as an option for treating drug-resistant bacterial infection related to beta-lactamase. However, these newly approved antibiotics are only active against SBLs, but not MBLs. Cefiderocol, a new siderophore cephalosporin, shows strong anti-bacterial activity against MBL-containing Enterobacteriacease, P. aeruginosa, and A. baumannii in vitro. It can form a complex with extracellular free iron and is transported through ECM to exert its anti-bacterial activity by inhibiting cell wall formation. Changes of amino acids through gene mutations in NDM-type β-lactamase result in a notorious NDM family containing various NDM-type β-lactamases variants, making the searching for a clinically relevant and effective NDM-type β-lactamase inhibitor more challenging.

Zhanel et al. (2019) established a mouse model of neurtropenic thigh and lung infection to evaluate anti-bacterial activity of cefiderocol in vivo, which was found to be effective against NDM-1-positive K. pneumoniae. In a rat model established by Matsumoto et al. (2017), it was found that cefiderocol can reduce NDM-1-positive K. pneumoniae in the lungs of an immunocompetent rat respiratory infection model more significantly than conventional ceftazidime monotherapy. However, these animal models cannot serve as a high-throughput detection and drug screening tool of numerous potential candidates for NDM-1 inhibitors. The detection and screening methods used in most of the existing in vitro studies are also susceptible to false-positive results due to the nature of beta-lactamase, which tends to form aggregates under aqueous conditions, thereby easily inducing non-specific inactivation of beta-lactamase during the high-throughput screening and detection in vitro.

Wang et al. (2015) proposed a multistep virtual screening method by using protein alignment and superposes biopolymer module in Molecular Operating Environment suit (MOE) or the Protein Model Portal (PMP) to analyze 22 reported NDM-1 X-ray crystallographic structures. Structures of different known beta-lactams or NDM-1 inhibitors obtained from ZINC database were docked into NDM-1 active site using different docking simulations in MOE and docking protocols (i.e., the docking box was first generated around the active site using the site finder module in MOE; the dimensions of the docking box were manipulated to accommodate all amino acid residues present in the active site). A virtual collection containing ~2.8 million drug-like compounds was taken from ZINC database as the screening library. The hits with firm binding conformations were collected and redocked into the active site. Selected compounds from the library were screened in 96-well plates using nitrocefin as substrate. Nitrocefin hydrolysis was monitored in the presence of NDM-1.

Furthermore, for patients with sepsis and septic shock, therapeutic drug monitoring (TDM) which is used to optimize the clinical effectiveness of beta-lactam antibiotics is very challenging as a routine practice because the commonly used instrumental method such as HPLC is very slow and complex in clinical practice.

Au et al. (2020) disclosed two biosensors based on two BADAN-conjugated beta-lactamases, E166Cb and E166Cb/N170Q, for beta-lactam antibiotic detection in vitro. An environment-sensitive BADAN probe was coupled onto location 166 at the active site of the PenP beta-lactamase E166C and E166C/N170Q mutants, respectively, to give fluorescence turn-on signals in response to beta-lactam antibiotics. Compared to fluorescein-modified counterparts, E166Cf and E166Cf/N170Q, the two biosensors based on BADAN-conjugated beta-lactamases, E166Cb and E166Cb/N170Q, respectively showed higher sensitivity, faster response in detecting moxalactam, and more stable fluorescence signals towards penicillin G.

Therefore, to facilitate high-throughput screening and detection of potential candidates of NDM-1 inhibitors, there is an unmet need for a reliable high-throughput screening tool that can identify active-site-binding candidates while at least diminishing or eliminating the drawbacks and problems in the existing screening methods or tools mentioned above.

### SUMMARY OF INVENTION

The present invention proposes labelling the active site of NDM-1 with an ultra-fast “light up” labelling agent, also known as an “active-site-labelling” strategy, for in vitro detection and high-throughput screening of potential candidates that can bind and inhibit the active site of NDM-1, which effectiveness can be verified by the restoration of bioactivity of certain carbapenem antibiotics against Gram negative bacteria such as E. coli and Klebsiella pneumoniae that contain MBLs.

Accordingly, a first aspect of the present invention provides a protein comprising a polypeptide having at least 95% sequence homology with an amino acid sequence selected from a group consisting of SEQ ID NOs: 1 to 5, wherein the amino acid at position 70 of SEQ ID NOs: 1-5 must be cysteine.

In certain embodiments, the polypeptide has at least 99% sequence homology with an amino acid sequence selected from a group consisting of SEQ ID NOs: 1 to 5.

In certain embodiments, the polypeptide has at least 99% sequence homology with SEQ ID NO: 1.

In certain embodiments, the polypeptide comprises SEQ ID NO: 1.

A second aspect of the present invention provides a biosensor comprising the protein according to the first aspect described herein and a fluorophore, wherein the fluorophore is covalently conjugated via an optional linker to a side chain of the cysteine at position 70.

In certain embodiments, the polypeptide has at least 99% sequence homology with an amino acid sequence selected from a group consisting of SEQ ID NOs: 1 to 5.

In certain embodiments, the polypeptide has at least 99% sequence homology with SEQ ID NO: 1.

In certain embodiments, the polypeptide comprises SEQ ID NO: 1.

In certain embodiments, the polypeptide is SEQ ID NO: 6.

In certain embodiments, the fluorophore comprises a fluorescein or BADAN.

In certain embodiments, the fluorophore comprises a moiety selected from the group consisting of:

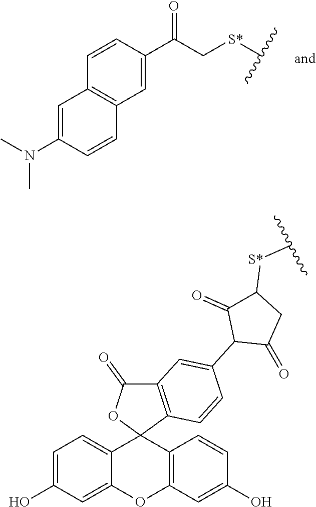

or a conjugate thereof, wherein S* represents the sulfur in the side chain of the cysteine at position 70.

A third aspect of the present invention provides a method for identifying a beta-lactam susceptible to hydrolysis by a New Delhi metallo-beta-lactamase, where the method comprises contacting the beta-lactam with the biosensor according to the first aspect described herein and detecting a change in fluorescence from the biosensor.

A fourth aspect of the present invention provides a method for identifying a beta-lactamase inhibitor, where the method comprises contacting the biosensor according to the first aspect described herein with a test compound and detecting a change in fluorescence from the biosensor.

A fifth aspect of the present invention provides a method for preparing the biosensor according to the first aspect described herein, where the method comprises:

providing a protein comprising a polypeptide having at least 95% sequence homology with an amino acid sequence selected from a group consisting of SEQ ID NOs: 1 to 5, wherein the amino acid at position 70 of SEQ ID NOs: 1-5 must be cysteine; and contacting the protein with a fluorescent labelling agent thereby forming the biosensor.

In certain embodiments, the polypeptide has at least 99% sequence homology with an amino acid sequence selected from a group consisting of SEQ ID NOs: 1 to 5.

In certain embodiments, the polypeptide has at least 99% sequence homology with SEQ ID NO: 1.

In certain embodiments, the polypeptide comprises SEQ ID NO: 1.

In certain embodiments, the fluorescent labelling agent comprises a fluorescein or BADAN.

In certain embodiments, the fluorescent labelling agent is fluorescein-5-malemide (F5M)

In certain embodiments, the protein and F5M are contacted in a molar ratio of 1:1-1.3, respectively.

Other aspects of the present invention include a kit or system for detection and high-throughput screening of potential candidates of beta-lactam or beta-lactamase inhibitor. The kit or system according to certain embodiments includes the biosensor described herein and components for qualitative or quantitative analysis of fluorescent signals emitted due to binding of any potential candidates to an active site of the fluorescent labelled NDM-1 enzyme. The present biosensor may also be used in evaluating clinical effectiveness of certain antibiotic treatments or therapeutic regimes involving use of certain antibiotics for treating infections arising from carbapenem-resistant bacteria such as MBL-containing Gram-negative bacteria.

Besides the site-specific mutation described in the foregoing aspects, the mutant of NDM-1 enzyme may also be prepared by site-specific mutation at other position than the 70^{th} amino acid residue from N-terminus of the mutant of NDM-1, including the 212^{th}, 219^{th}, or 223^{rd} amino acid residue. Similarly, the original amino acid residue will be mutated into cysteine for subsequent site-specific labeling of fluorophore.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1A depicts a general scheme of beta-lactam hydrolysis by beta-lactamase;

FIG. 1B schematically depicts a 3-D structure of NDM-1 protein backbone with helices and strands, and the two zinc ions at the active sites are shown as spheres;

FIGS. 2A-2H show molecular mass of the NDM-1 mutant with or without fluorescein labeling, namely F70C and F70Cf, determined by LC-ESI-MS and SDS-PAGE analyses: (2A) The molecular mass of NDM-1 mutant, F70C, prepared according to certain embodiments; (2B) The molecular mass of fluorescein-labelled NDM-1 mutant, F70Cf, prepared according to certain embodiments which were incubated with a 1.3-fold molar excess of fluorescein-5-maleimide at different time intervals; (2C) The molecular mass of the F70Cf prepared according to other embodiments which were incubated with a 3-fold molar excess of fluorescein-5-maleimide at different time points; (2D) The molecular mass of wild-type NDM-1; (2E-2F) SDS-PAGE analysis of purified F70C: M, protein markers; TL, total lysate; S/N, supernatant total protein; FT, flow through; 10%, 10% of elution buffer; 20%, 20% of elution buffer; 30%, 30% of elution buffer; 40%, 40% of elution buffer; 60%, 60% of elution buffer; (2G) A protein gel stained with Coomassie Blue; (2H) The same protein gel as in FIG. 2G illuminated with ultraviolet light;

FIG. 3 shows secondary structure of F70C, F70Cf and wild-type NDM-1 by circular dichroism (CD) spectrometry analysis;

FIGS. 4A-4C shows an electrospray ionization mass spectrometry (ESI-MS) spectra of trypsin digested F70C and F70Cf: (4A) The peptide fragment showing the amino acid sequence from the positions of 53 to 81 (QLAPNVWQHTSYLDMPGCGAVASNGLIVR) with a m/z value of 1033.1849 indicates the position of cysteine-70 (target mutation site, highlighted in dark gray) while the fragment showing the amino acid sequence from the positions of 182 to 211 displays the position of cysteine-208 (active site, highlighted in light gray); (4B) The m/z values of peptide fragments of cysteine-70 conjugated with F5M (highlighted in dark gray) and cysteine-208 conjugated with F5M (light gray) are 1181.5397 and 1170.5363, respectively; (4C) positions of two key cysteine residues on two peptide fragments with amino acid sequences from positions 53 to 81 and from positions 182 to 211 of the mutant of the NDM-1 enzyme (indicated by two dotted line rectangular boxes), respectively, which were prepared by trypsin digestion of the NDM-1 mutant according to certain embodiments, where cysteine residue at position 70 (C70) in the fragment from positions 53 to 81 representing a specific site of mutation and another cysteine residue at position 208 (C208) in the fragment from positions 182 to 211 representing a buried cysteine residue in the active binding pocket of the NDM-1 mutant are indicated by dark gray and light gray boxes, respectively;

FIGS. 5A-5B show time-course fluorescence measurements from F70Cf in a mole ratio of 1:0 (baseline), 1:150, and 1:1500 to two tested antibiotics: (5A) cefotaxime; and (5B) cephalothin;

FIGS. 6A-6B show fluorescence microplate reader measurements F70Cf in a mole ratio of 1:1500 to two tested cefotaxime and cephalothin antibiotics;

FIGS. 7A-7E show changes of fluorescent signals from fluorescent labelled NDM-1 according to certain embodiments under exposure to different concentrations of the following NDM-1 inhibitors, respective: (7A) L-captopril; (7B) D-captopril; (7C) DL-thiorphan; (7D) Thanatin; and (7E) L-arginine;

FIGS. 8A-8F show fluorescence measurements of F70Cf with various inhibitors using a fluorescence microplate reader: the fluorescence profiles for (8A) L-captopril, (8B) D-captopril, (8C) DL-thiorphan, (8D) thanatin, (8E) L-arginine, and (8F) Tris-HCl buffer (50 μM ZnC12, 20 mM, pH7.0); the mole ratio of F70Cf to each inhibitor is 1:1500

FIGS. 9A-9F show the results of binding studies of F70Cf with different inhibitors by native ESI-MS, where E and EI represent F70Cf and F70Cf-inhibitor complexes, respectively: (9A) The molecular mass of F70Cf in native state was measured to be 26189 Da; the binding profiles for (9B) L-captopril, (9C) D-captopril, (9D) DL-thiorphan, (9E) thanatin, and (9F) L-arginine;

FIGS. 10A-10B schematically depict how the (10A) L-captopril and (10B) Thanatin depicted in FIGS. 7A and 7D, respectively, change the 3-D conformation of the active binding site of the fluorescent labelled NDM-1 and activate the fluorescent signal emission, accordingly; protein traces are shown as cartoon models in grey; zinc ions are shown as spheres; apo model was constructed from PDB ID 3SPU (B chain) whereas the L-captopril complex schematic in FIG. 10A was from PDB ID 4EXS (B chain); the NDM-1: thanatin complex schematic in FIG. 10B was created with the help of ColabFold (“AlphaFold2 with MMSeq2” notebook, using the “PDB70” template); both FIGS. 10A and 10B were created with PyMOL;

FIG. 10C shows fluorescence spectra of F70Cf in the presence of trypsin recorded at different time intervals;

FIG. 11 schematically depicts a method of detecting and screening potential candidates of NDM-1 inhibitors from compound library using the present biosensor according to certain embodiments.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

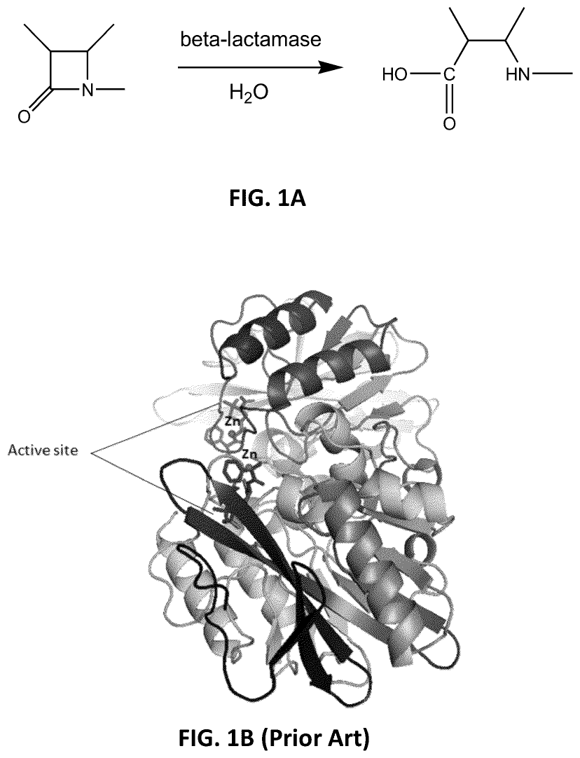

To date, more than 400 clinically relevant β-lactamases have been identified. Such bacterial enzymes can efficiently open the four-membered β-lactam ring through hydrolysis, thus making the antibiotics clinically inactive (FIG. 1A). A catalytic pathway of β-lactamases towards β-lactam antibiotics can be generalized in the following scheme:

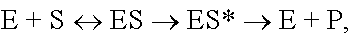

where E is the free enzyme (β-lactamase), S the substrate (β-lactam antibiotic), ES the non-covalent enzyme-substrate complex, ES* the covalent enzyme-substrate complex, and P the carboxylic acid product.

Metallo-beta-Lactamases (MBLs) belong to class B β-lactamases that hydrolyze almost all clinically-available β-lactam antibiotics. They all have the αβ/βα sandwich fold of the metallo-hydrolase/oxidoreductase superfamily. MBLs possess a shallow active-site groove with one or two divalent zinc ions, bordered by flexible loops. NDM-1 belongs to subclass B1 (there are three subclasses namely B1, B2 and B2 under class B) of MBLs with two conserved identical coordinating residues (His-His-His) and (Asp-Cys-His) coordinated with two zinc ion binding sites, where the first zinc ion binding site (Zn1) is responsible for coordinating His120, His122 and His189 residues, while the second zinc ion binding site (Zn2) is coordinated with Asp124, Cys208 and His250. The two zinc ions are bridged by a water molecule, acting as a nucleophile during beta-lactam hydrolysis to attack atom C of beta-lactam. The 3D structure of NDM-1 with an active site and two zinc molecules is shown in FIG. 1B (by Khan et al., 2017).

Liang et al. (2011) suggested that an active site of NDM-1 has two mobile loops, in which loop1 (or called flapping loop) is composed of amino acids LDMPGFGAVA (residues 65-74), which creates a larger active site cavity with less bulky amino acids compared to the corresponding loop1 of other MBLs, while loop2 has Ala215 to enlarge active site cavity. The less bulky amino acids in the active site, such as glycine, could make the active site of NDM-1 more flexible for substrate binding. The N-terminus of NDM-1 is also longer than other analogues of MBLs. Overall, these unique structural characteristics likely contribute to a higher potency and a broader range of substrate binding and hydrolysis of beta-lactams, especially for those bulkier antibiotics.

In the last few years, 17 new variants of NDM-1 have been evolved by changing one or two residues at different positions. The emergence of bacteria carrying such NDM-1 encoding genes represent a big challenge for physicians to treat infected patients. Many NDM-1 variants evolved in Enterobacteriaceae, Vibrionaceae and other non-fermenters by single and double amino acid residue substitutions at different positions, for example, NDM-1 (major variant), NDM-2, NDM-3, NDM-4 and NDM-5 (minor variants) (represented by SEQ ID NOs: 1-5, respectively), have been reported worldwide. The gene encoding NDM-1 is carried by plasmids and hence easily moves to other microorganisms via horizontal gene transfer, thereby increasing the probability of emergence of drug resistant strains of pathogenic microorganisms. A number of variants of NDM-1 have been reported. For example, NDM-2 had a substitution of Cysteine to Glycine at position 82, and amino acid being substituted by alanine at position 28 in place of proline, in A. baumannii. Another variant NDM-3 with an amino acid substitution of Aspartate to Asparagine at position 95 was observed in E. coli. On the other hand, NDM-4 variant showed substitution of amino acid from 154^{th} Methionine to Leucine in E. coli (Khan et al., 2017).

Therefore, in order not to affect the active site binding activity of NDM-1 enzyme, the present invention proposes selecting an amino acid (more exposed) close to the active site with a cysteine residue and then conjugating a thiol-reactive fluorophore to such cysteine residue, in which the fluorophore can be specifically conjugated to the selected cysteine residue and fluorescently sense the binding of inhibitors or antibiotics against NDM-1. The wild-type NDM-1 β-lactamase itself has a cysteine residue at the 208^{th} position (C208) which is responsible for binding to Zn²⁺ for catalyzing the hydrolysis of β-lactam antibiotics. C208 is buried within the active site, such that fluorophore labelling at C208 is more difficult due to its binding to Zn2+ and the buried environment around C208. These structural properties largely prevent unwanted labelling C208 with a thiol-reactive fluorophore at C208, which would otherwise lead to the loss of the hydrolytic activity of NDM-1.

Accordingly, at the first place, the 70^{th} position which is originally phenylalanine (F) (F70) is selected as a target of site-directed mutation into a cysteine residue (C70) for subsequent site-specific labeling of said thiol-reactive fluorophore. In certain embodiments, the thiol-reactive fluorophore includes thiol-reactive fluorescein. More specifically, the thiol-reactive fluorescein comprises fluorescein-5-maleimide (F5M). In other embodiments, the fluorophore can be BADAN. In the embodiments using F5M as the site-specific labeling of C70, a carefully selected mole ratio between the NDM-1 enzyme and F5M should be used to avoid non-specific conjugation to other cysteine residue in the active binding pocket such as C208. In certain embodiments, the mole ratio between the NDM-1 enzyme and F5M is 1:1-1.3. More specifically, the NDM-1 enzyme and F5M have a mole ratio of 1:1.3. In certain embodiments, the mole ratio between NDM-1 enzyme and F5M being 1:3 or higher will result in non-specific conjugation to more than one cysteine residues.

Overall, the proposed strategy (the “active-site-labelling” strategy) of the present invention is that at least one type of fluorophore such as fluorescein or BADAN is conjugated to at least one amino acid residue near the active binding site of a mutant of NDM-1 enzyme (e.g., F70C, D212C, G219C, D223C) so as to enable the fluorescent labelled NDM-1 enzyme to fluorescently respond to a potential drug binding event whilst the catalytic activity of the NDM-1 enzyme is maintained. In addition, the fluorescent labelled NDM-1 enzyme can also be a natural molecular target for antibiotic detection. Since fluorescent signals can be qualitatively and quantitatively analyzed, the present biosensor based on the fluorescent labelled NDM-1 enzyme is also useful in evaluating clinical effectiveness of certain beta-lactam antibiotics.

Details of how the present biosensor is designed and fabricated, and its characteristics as to binding activity, fluorescence response mechanism, and applications (practical and potential) will be provided as examples and various embodiments described hereinafter. It should be understood that the following examples should be non-limiting and for the purpose of assisting the illustration and understanding of the present invention. Scope of the present invention should be referred to the appended claims.

### EXAMPLES

### Example 1—Synthesis and Characterization of Fluorescent Labelled NDM-1

The NDM-1 coding sequence was cloned into a pET-3k vector, where the pET-3k vector was a modified pET-3a vector with the replacement of ampicillin by kanamycin. The NDM-1 mutant (F70C) was constructed by the replacement of phenylalanine located at 70^{th} position by a cysteine using QuikChange Site-Directed Mutagenesis Kit (Stratagene, CA, USA). The mutagenic forward and reverse primers are 5′ GAC ATG CCG GGT TGC GGG GCA GTC GCT TCC 3′ (SEQ ID No: 7) and 5′ GGA AGC GAC TGC CCC GCA ACC CGG CAT GTC 3′ (SEQ ID No: 8), respectively.

For protein expression of wild-type NDM-1 and its mutant (F70C) proposed in the present invention, a single colony of E. coli BL21 (DE3) inserted with a designed vector was incubated into 10 mL of Lysogeny broth (LB) medium with 50 g/mL kanamycin and cultured at 30° C. with shaking at 250 rpm overnight. 2 mL of this LB medium was transferred into a 200 mL LB medium. When OD₆₀₀ of the culture reached 0.6, isopropylthiogalactoside (IPTG) with a final concentration of 0.2 mM was added and incubated at 16° C. at 250 rpm overnight.

For protein purification, to avoid any unwanted divalent metal ions, such as nickel (II) and cobalt (II) ions, which may be inserted into metalloprotein NDM-1 during the purification process that may affect the downstream assays, no purification tag including (His)₆-tagged was used. Cells were harvested by centrifugation at 8,000 rpm at 4° C. for 30 min. The cell pellet was resuspended in lysis buffer (50 μM ZnCl₂, 20 mM Tris-HCl buffer, pH 7.4) and lysed by an ultrasonic homogenizer (QSonica sonicators). The supernatant was harvested by centrifugation at 13,000 rpm at 4° C. for 1 h. The filtered supernatant was applied into HiTrap Q HP (GE Healthcare) column with washing buffer (20 mM Tris-HCl buffer, pH 7.4) and elution buffer (1 M NaCl, 20 mM Tris-HCl buffer, pH 7.4). Impurities were removed in flow-through and targeted proteins were collected in 10% elution. The targeted proteins were buffer exchanged into labelling buffer (50 μM ZnCl₂, 20 mM Tris-HCl buffer, pH 7.0) and stored at −80° C.

For specifically labeling F70C with a thiol-reactive fluorescein to generate fluorescein-labelled F70C (F70Cf), the F70C enzymes were initially diluted into 1 mg/ml by labelling buffer. Different folds of molar excess (e.g., 1:1.3 and 1:3) of fluorescein-5-maleimide (F5M) were added to the enzyme solution. The mixtures were reacted at room temperature (22° C.) with different time points (from 30 min to 2 h). Amicon (MWCO=10k Da) was used to remove excess dye using labelling buffer. All labelled F70C (F70Cf) enzymes were stored at −80° C.

In other embodiments, a fluorophore such as fluorescein or BADAN could be conjugated to other substituted amino acid residue with cysteine than F70C which also does not affect the binding activity of the active site of NDM-1 enzyme whilst being theoretically fluorescently responsive to the change in physiological condition due to the binding of the active site. That other substituted amino acid residue may be D212C, G219C or D223C.

Molecular mass, purity and labelling efficiency of F70Cf were also determined by using liquid chromatography-electrospray ionization mass spectrometry (LC-ESI-MS) (Agilent 6540 QTOF mass spectrometer coupled with an Agilent 1290 Infinity UHPLC system). Purified wild-type NDM-1 enzymes, F70C enzymes and labelled F70Cf enzymes were injected into a C4 LC column and eluted with a linear gradient from 95% solvent A: 5% solvent B to 5% solvent A: 95% solvent B, where solvent A was milliQ water and solvent B was acetonitrile, each with 0.1% formic acid. The mass spectrometer was operated in a positive ion mode. ESI-MS data was acquired with a m/z range of 600-1600, from which multiply-charged mass spectra were obtained. The multiply charged mass spectra were deconvoluted by the MassHunter BioConfirm program to obtain the molecular mass of proteins. The calculated molecular weight of NDM-1 F70C, NDM-1 F70Cf and wild-type NDM-1 were 25637 Da, 26064 Da and 25681 Da, respectively. Since there are two cysteine residues in F70C (one is buried inside the active site (C208) and bound to Zn2+, whereas the other (C70) lies close to the active site but with higher exposure to the external aqueous environment), it is important to optimize the fluorophore labelling ratio so as to avoid unwanted labelling at C208 (at the active site).

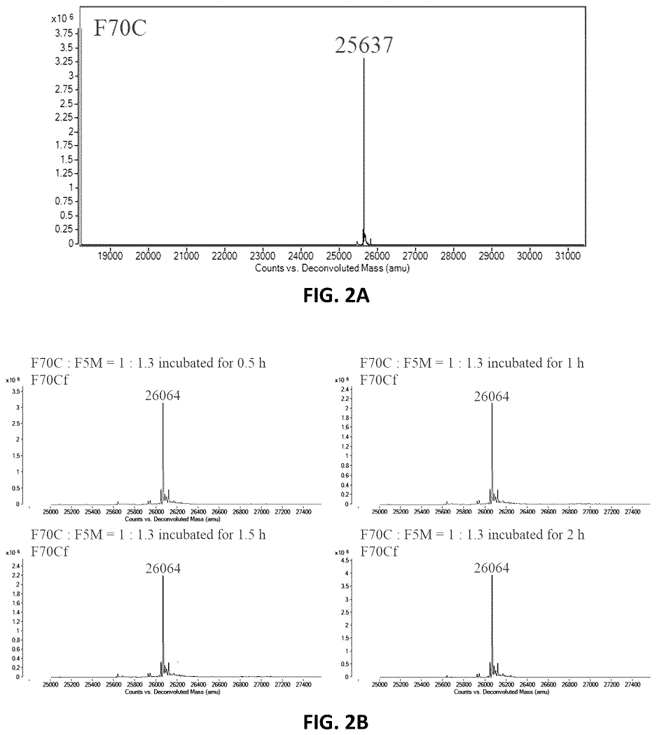

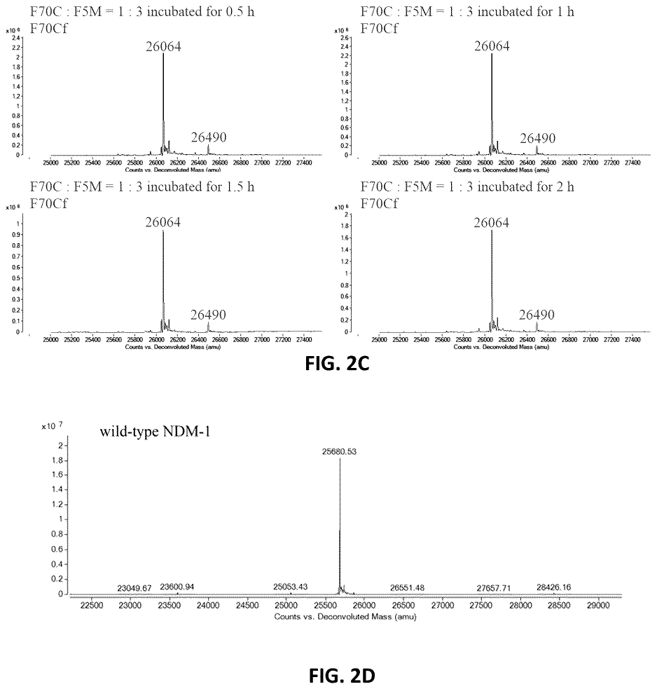

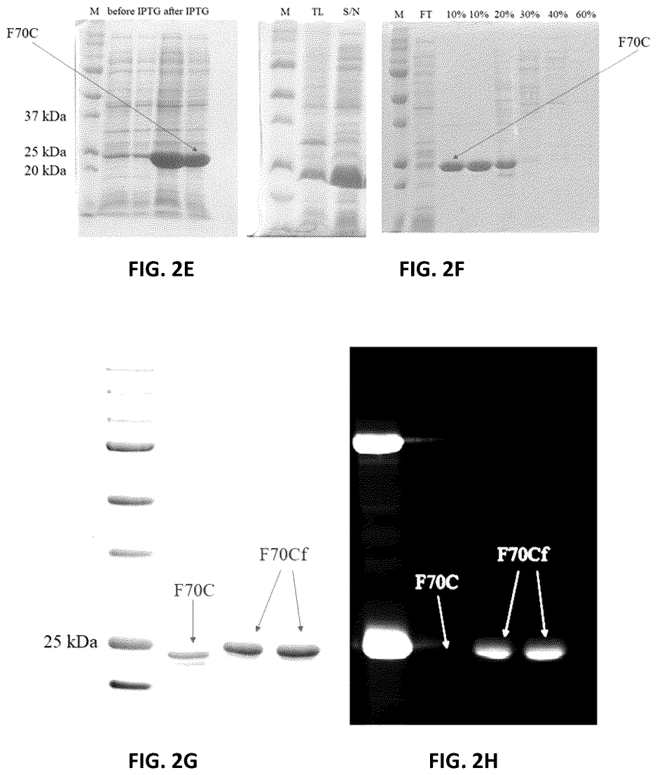

Compared to a higher mole ratio of fluorescein (1:3), it was observed that labelling F70C to F5M in a mole ratio of 1:1.3 resulted in site-specific labelling at C70 only, presumably due to high exposure of F70C to the external aqueous environment and the buried environment around C208 (also bound to Zn2+) within the active site making F5M difficult to approach C208 (FIG. 2B). In the case where the mole ratio of F70C to F5M was increased to 1:3, fluorophore labelling at both C70 and C208 occurred (FIG. 2C, molecular mass=26490 Da). These measurements in terms of molecular mass are consistent with the calculated molecular mass of F70C and F70Cf (25636 and 26063 Da, respectively, calculated by the Expasy program). Moreover, the mass difference between the labelled and unlabelled F70C mutants was consistent with the molecular mass of F5M (~427 Da).

In order to study the action of naturally occurring Zn²⁺ on β-lactam antibiotics and inhibitors, wild-type NDM-1 (with a measured molecular mass of 25681 Da as shown in FIG. 2D) and F70C mutant without (His)₆-tags were produced to avoid any replacement of other divalent cations, such as Ni2+ and Co²⁺, during purification. F70C (with a molecular mass of 25637 Da as shown in FIG. 2A) was highly expressed in E. coli after the induction of IPTG (FIG. 2E) in a high purity by using anion exchange column in 10% elution buffer (FIG. 2F). FIGS. 2G and 2H further show that F70Cf emitted fluorescence signals under UV light on a SDS-PAGE gel.

The specificity of fluorescein labelling site of F70C at 1.3-fold molar excess was further studied by trypsin digestion of F70Cf, which was analyzed by ESI-MS. Labelling site of F70Cf was determined by initially subjecting F70C and F70Cf enzymes to buffer-exchange in 50 mM ammonium bicarbonate and subsequently adding sequencing grade trypsin (Progema V5111) into the isolated enzymes in a ratio of trypsin:enzyme 1:50 (wt/wt). Protease digestions were conducted at 37° C. for 16 h. Digested F70C and F70Cf enzymes were analyzed by an Agilent 6540 QTOF mass spectrometer coupled with an Agilent 1290 Infinity UHPLC system. Peptides were separated and eluted in a C18 LC column with a linear gradient elution from 95% solvent A: 5% solvent B to 5% solvent A: 95% solvent B, where solvent A was milliQ water and solvent B was acetonitrile, each with 0.1% formic acid. Peptide assignment was performed with the Agilent Masshunter-BioConfirm software.

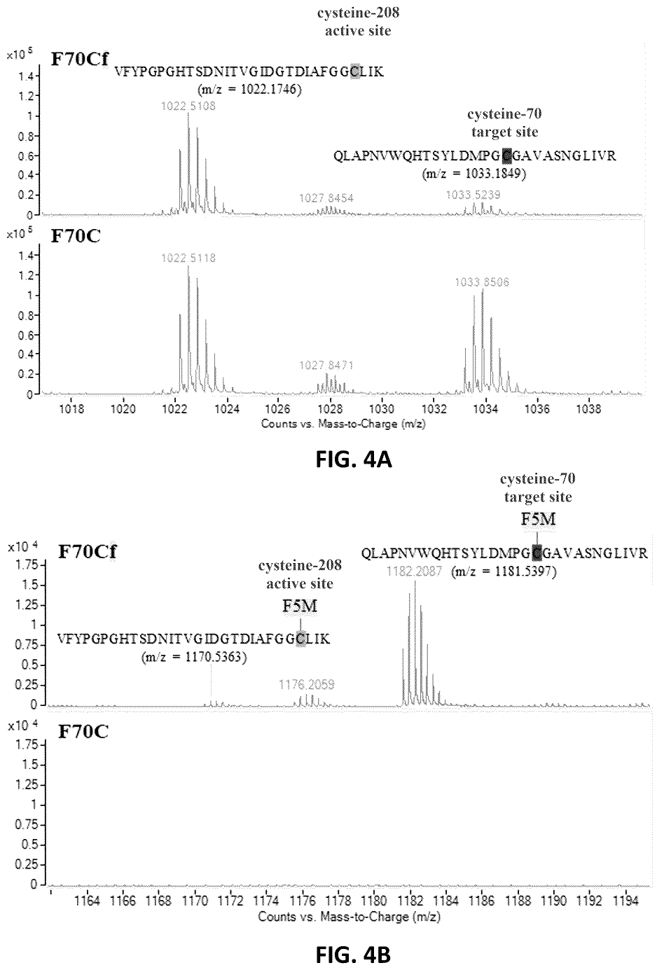

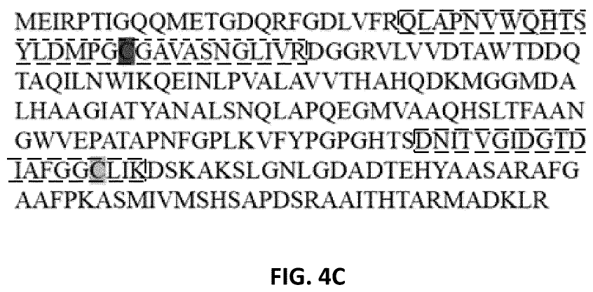

After trypsin digestion, two peptide fragments including C208 (from positions 182 to 211 of SEQ ID NO: 6: VFYPGPGHTSDNITVGIDGTDIAFGGCLIK) with mass to charge ratio (m/z) of 1022.1746 and C70 (from positions 53 to 81 of SEQ ID NO: 6: QLAPNVWQHTSYLDMPGCGAVASNGLIVR) with a m/z of 1033.1849 were obtained, respectively (the two amino acid sequences of the digested fragments are also shown in FIG. 4C). Moreover, m/z of digested peptide fragments conjugated with F5M at C208 and C70 were 1170.5363 and 1181.5397, respectively. The ESI-MS spectra from FIGS. 4A and 4B further suggest that virtually all the F5M was labelled at C70 at the mole ratio of 1:1.3, which was revealed by the dominant peak. Only a minor mass peak was associated with C208 attached to F5M, proving a highly specific fluorescein labelling to C70 in NDM-1 mutant.

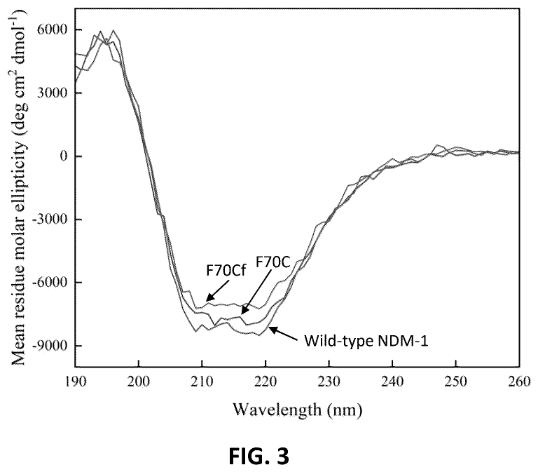

Secondary structure of F70C, F70Cf and wild-type NDM-1 was analyzed by circular dichroism (CD) using JASCO J-1500 Circular Dichroism Spectrophotometer to determine their secondary structure under 10 mM potassium phosphate buffer at pH 7.5 with final concentration 5 μM. Each CD spectrum was collected from an average of 3 scans and the CD signals were expressed as molar ellipticity (deg cm² dmol⁻¹). The results are shown in FIG. 3. From the CD spectra in FIG. 3, F70C shows similar secondary structure and specific activity to those of the wild-type NDM-1 against different β-lactam antibiotics, indicating that the F70C mutation does not significantly weaken the activity as compared to wild-type NDM-1.

Specific activity (U/mg) of wild-type NDM-1, F70C and F70Cf against different antibiotics were measured by ultraviolet-visible spectroscopy. Enzymes (final concentration: 0.001 mg/ml) were incubated with 100 μM of different antibiotics: cefotaxime, ceftazidime, penicillin G, ampicillin and meropenem, in 500 μL buffer (50 μM ZnCl₂, 20 mM Tris-HCl buffer, pH 7.0). Antibiotic hydrolysis was monitored at 264 nm (cefotaxime), 260 nm (ceftazidime), 240 nm (penicillin G), 235 nm (ampicillin) and 297 nm (meropenem). The molar extinction coefficients were used as follows: cefotaxime=−7250 M⁻¹ cm⁻¹, ceftazidime=−8660 M⁻¹ cm⁻¹, penicillin G=−560 M⁻¹ cm⁻¹, ampicillin=900 M⁻¹ cm⁻¹ and meropenem −10940 M⁻¹ cm⁻¹. The results are summarized in Table 1.

TABLE 1

| | Specific activity (U/mg) | | |
|---|---|---|---|
| Antibiotics | Wild-type NDM-1 | F70C | F70Cf |
| Cefotaxime | 8166 ± 937 | 8195 ± 1960 | 1529 ± 121 |
| Ceftazidime | 3446 ± 150 | 3835 ± 216 | 308 ± 33 |
| Penicillin G | 54764 ± 3013 | 60854 ± 1436 | 10720 ± 330 |
| Ampicillin | 16349 ± 880 | 13880 ± 1406 | 779 ± 114 |
| Meropenem | 6834 ± 1582 | 5863 ± 291 | 506 ± 30 |

### Example 2-Fluorescence Response and Binding Mechanism of Fluorescein-Labelled NDM-1 with Respect to Various NDM-1 Inhibitors

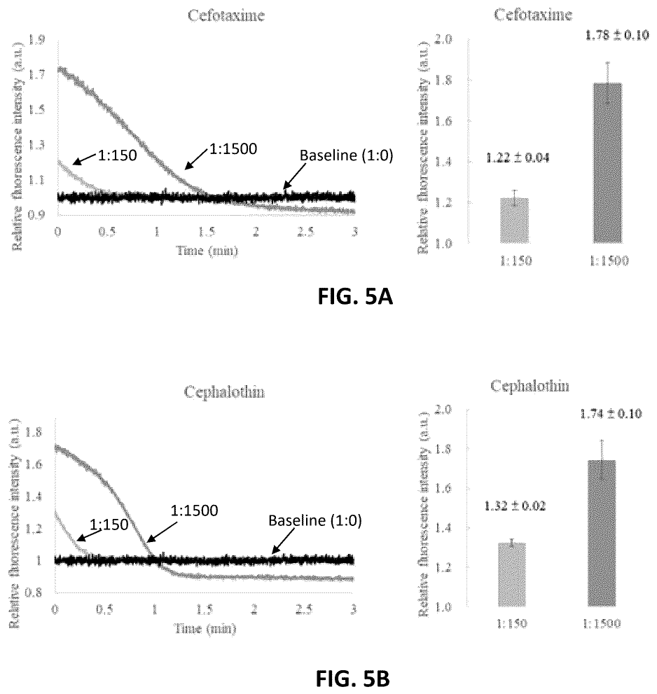

To investigate fluorescence response of the proposed NDM-1 mutant to two existing β-lactam antibiotics, fluorescein-labelled NDM-1 mutant, F70Cf, was mixed with cefotaxime and cephalothin in two concentrations (in mole ratios of 1:150 and 1:1500) and compared the fluorescence intensity level of these two samples with a blank (i.e., no cefotaxime nor cephalothin, or mole ratio=1:0) under a spectrofluorometer. As seen from FIGS. 5A and 5B, fluorescence signals increased and then decreased gradually as a function of time. It has been shown in other previous studies (Chan et al., 2008; Tsang et al., 2011) that binding of β-lactam antibiotics to the enzyme's active site induces the antibiotic to depart from the active site, so that the fluorescein label (F5M) becomes well exposed to external aqueous environment and/or stays away from amino acid quenchers in the active site. When the antibiotic is hydrolyzed to the acid product, the fluorescein label (F5M) returns to the active site, thus restoring its weak fluorescence signal.

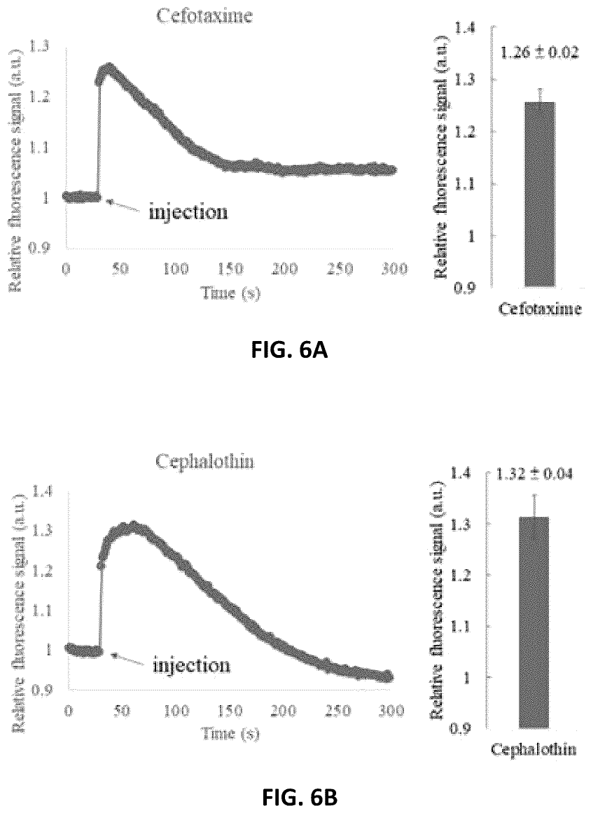

To investigate compatibility of F70Cf with a high-throughput screening platform, a F70Cf was initially added to a fluorescence microplate reader followed by injection of 1:1500 cefotaxime and cephalothin, respectively. As seen from FIGS. 6A-6B, both cefotaxime and cephalothin in mole ratio of 1:1500 activated F70Cf to give stronger fluorescence signals and then declining fluorescence signals as a function of time, similar to the results recorded by spectrofluorometer as shown in FIGS. 5A-5B. These results suggest the compatibility and capability of F70Cf in in vitro drug screening with a high-throughput microplate reader.

To study the ability of F70Cf to detect non-β-lactam inhibitors (e.g., L-captopril and DL-thiorphan which were obtained from Sigma-Aldrich (St. Louis, MO); thanatin (with an amino acid sequence of GSKKPVPIIYCNRRTGKCQRM) was purchased from GL Biochem (Shanghai, CN); and D-captopril which was bought from TLC Pharmaceutical Standards (Pony Dr, CA)), Fluorsecein-5-maleimide was obtained from Invitrogen (Carlsbad, CA, USA)) which are able to bind to NDM-type β-lactamases, fluorescence measurements for the detection of F70Cf against those inhibitors were performed on Agilent Cary Eclipse Fluorescence Spectrophotometer (Agilent). Time-course fluorescence measurements were recorded at 518 nm by exciting the F70Cf (final concentration: 0.13 μM) at 494 nm with different concentrations of the inhibitors (0, 2, 20 and 200 μM, respectively) in labelling buffer (50 μM ZnCl₂, 20 mM Tris-HCl buffer, pH 7.0) in a quartz cuvette. Triplicate measurements were performed for each inhibitor. L-arginine in 200 μM was also used as a control.

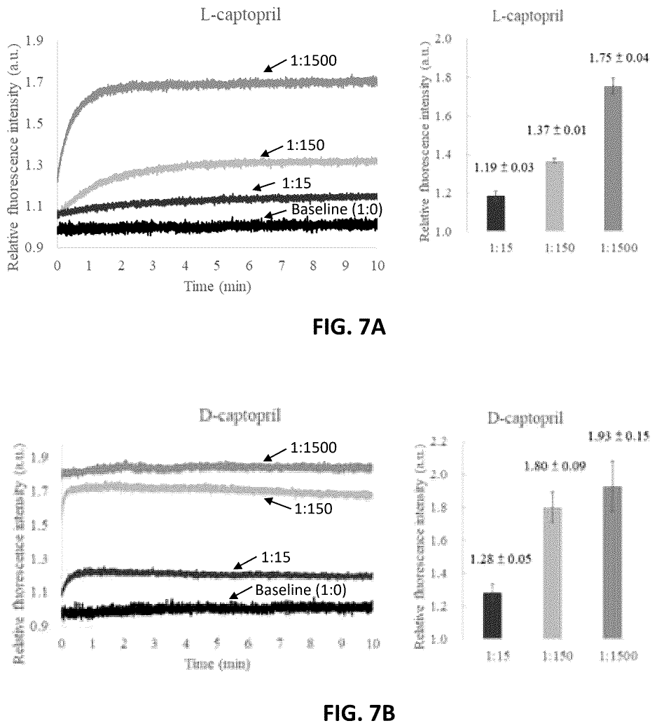

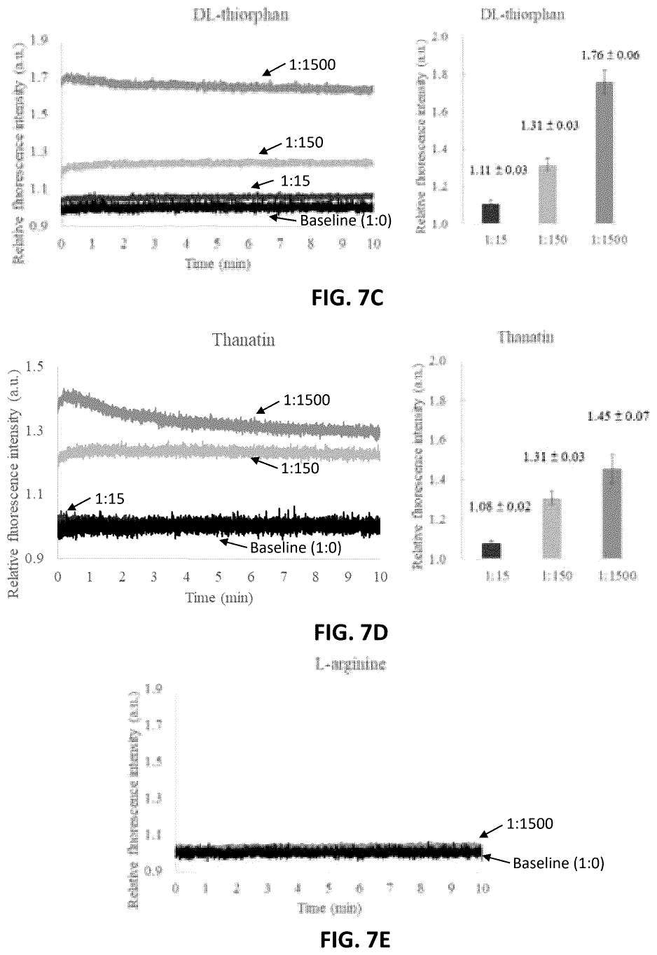

As seen from FIGS. 7A-7D, the fluorescence signals of F70Cf increased with an increasing dose of the inhibitors. The sustained fluorescence increases suggest that the inhibitors continuously occupy the active site of F70Cf. Among the inhibitors, L-captopril showed a gradual increase in fluorescence in the initial phase, whereas D-captopril, DL-thiorphan and thanatin virtually did not (FIGS. 7A-7D, respectively). These findings indicate that D-captopril, DL-thiorphan, and thanatin bind much faster to F70Cf as compared L-captopril. FIG. 7E shows no fluorescence signal from F70Cf when 1:1500 L-arginine was added. From these results, it can be suggested that the fluorescence response of F70Cf to these inhibitors is given in a dose-dependent manner.

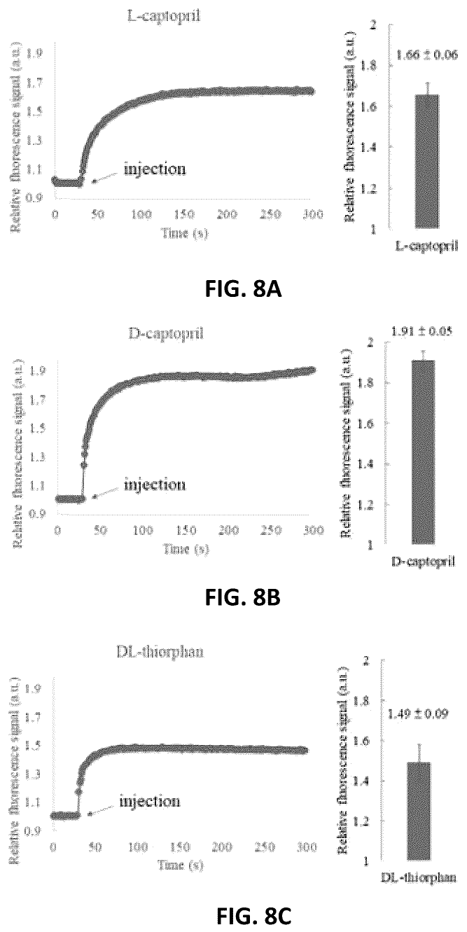

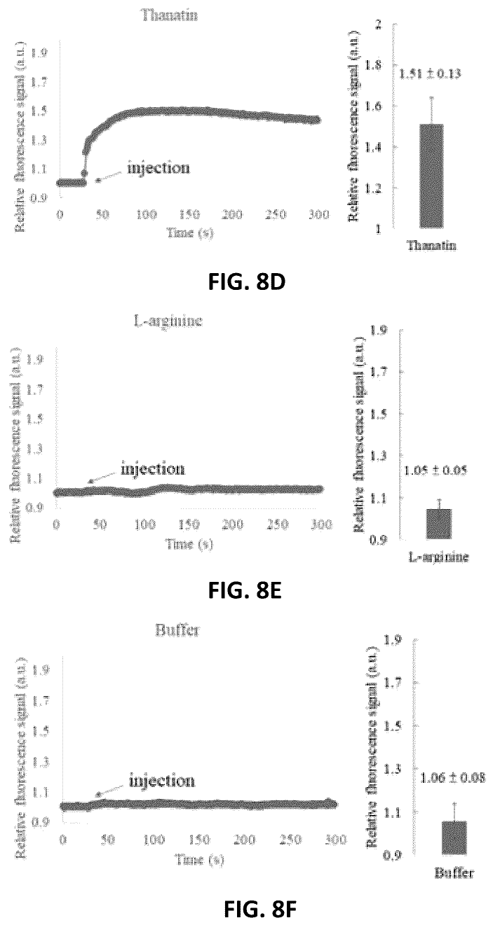

To trace the initial binding phases of the fast inhibitors (D-captopril, DL-thiorphan, and thanatin), a microplate reader was used to monitor the fluorescence signals as a function of time. As seen in FIGS. 8A-8D, the initial increases in fluorescence signal for D-captopril, DL-thiorphan, and thanatin were much faster than that of L-captopril, suggesting that D-captopril, DL-thiorphan, and thanatin are likely to have higher binding affinity to F70Cf than L-captopril. These results are consistent with the IC₅₀ values of these inhibitors to NDM-1 in some previous studies, in which the IC₅₀ values of L-captopril, D-captopril, DL-thiorphan, and thanatin were found to be 157.4, 20.1, 1.8, and 3.21 μM, respectively (Brem et al., 2016; Klingler et al., 2015; Ma et al., 2019). These observations reveal that F70Cf is capable of screening for non-covalent small molecules/peptides in vitro. To verify whether F70Cf is specific to active-site binders only, buffer and L-arginine were used as control and the results from FIGS. 8E and 8F show no fluorescence signals from F70Cf against these non-binders.

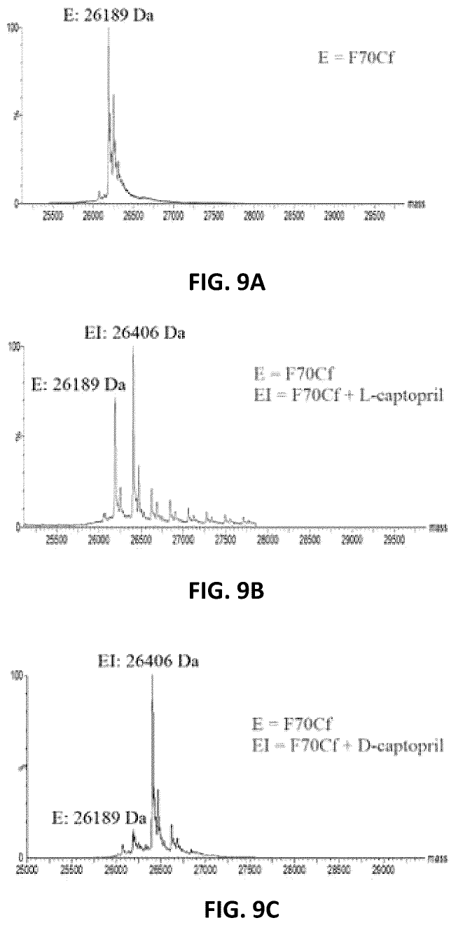

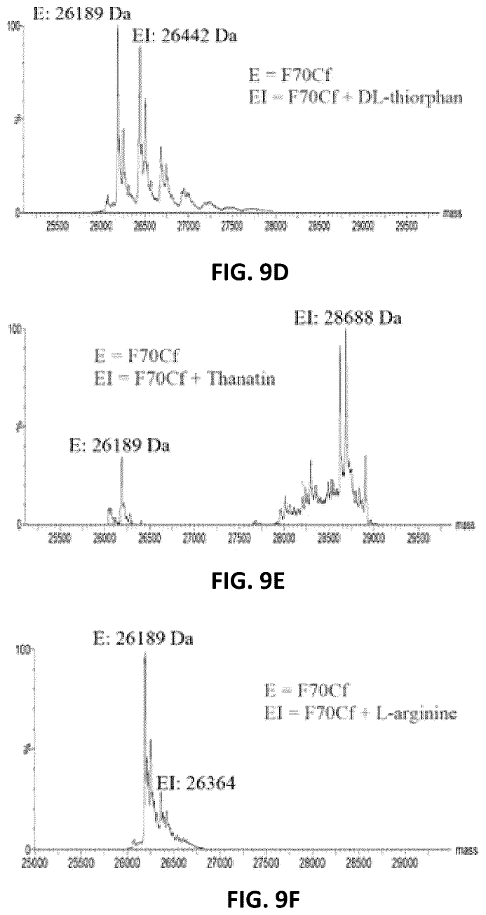

To further verify that F70Cf can bind to these non-β-lactam inhibitors non-covalently, native ESI-MS measurements were performed. The interactions between intact F70Cf and the inhibitors (L-captopril, D-captopril, DL-thiorphan and thanatin) in various concentrations (i.e., 0, 1:15, 1:150 and 1:1500) were analyzed by using native ESI-MS on a Waters Synapt G2-Si Quadrupole-Ion Mobility-Time-of-flight Mass Spectrometer. Prior to ESI-MS analysis, protein was buffer-exchanged into 20 mM ammonium acetate with 50 μM ZnCl₂ buffer with ultra-filtration devices. Equal volumes of F70Cf and inhibitors were mixed in a molar ratio of 1:20 for 10 minutes, and the mixture was directly loaded into metal-coated glass capillaries, which were subsequently mounted onto the nano-ESI source for MS analysis to test the fluorescence signal performance of the fluorescent labelled NDM-1. The results are shown in FIGS. 9A-9F. From the results in FIGS. 9A-9F, it is suggested that the inhibitors can form non-covalent complexes with F70Cf in a significant population, whereas the non-binder L-arginine virtually cannot form a F70Cf-L-arginine complex in a significant population. These results align with the findings from FIGS. 7A-7D and 8A-8D that the fluorescence enhancements of F70Cf with the inhibitors arise from the binding of the inhibitors to the active site.

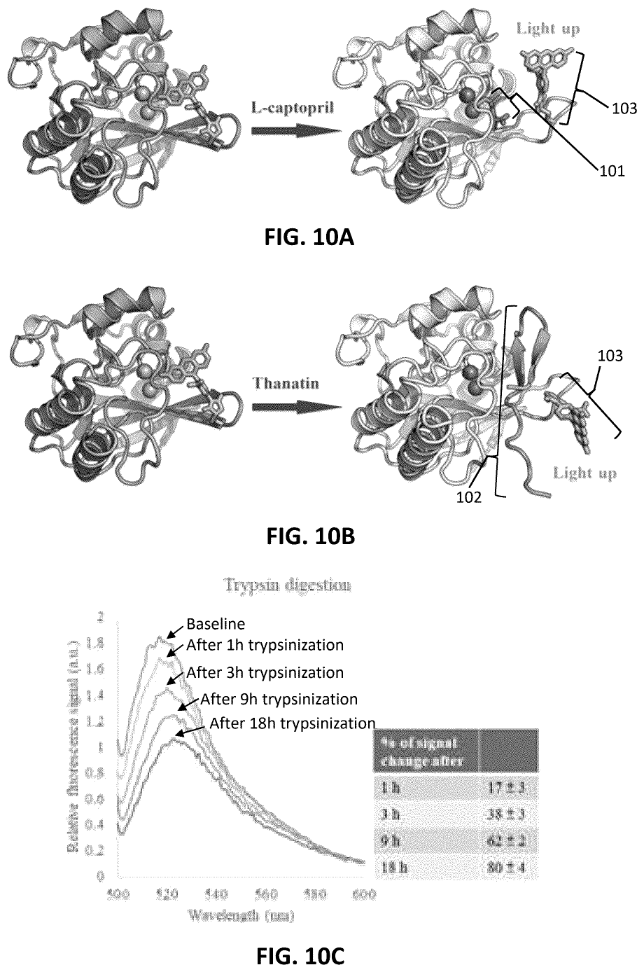

To understand the mechanism of F70Cf fluorescence enhancement after binding to inhibitors, molecular modeling was used. FIGS. 10A-10B schematically depict how the two inhibitors, L-captopril and thanatin (101, 102), turn on the fluorophore 103 of fluorescent labelled NDM-1 (“light up”) to emit fluorescence upon exposure in terms of the conformational change in the molecular structure by a molecular modeling of F70Cf with these two inhibitors. The two schematics in the left column of FIGS. 10A and 10B show the 3-D structure of the F70Cf before exposure to the two positive controls, which was created with Coot and JLigand of the CCP4 suite, while the two schematics in the right column show the 3-D conformational change in the F70Cf after exposure to the two positive controls. In the schematics of FIGS. 10A and 10B, the F70Cf has the fluorophore-labelled C70 sidechain (in grey) buried and occupying the substrate-binding site, which experiences a hydrophobic environment that may also contain amino acid quenchers, resulting in a weaker fluorescence signal (left panel of FIG. 10A). When L-captopril 101 binds to the F70Cf active site, it displaces the fluorescein label attached to C70 into the external aqueous environment (103), leading to a stronger fluorescence signal (right panel of FIG. 10A). When thanatin 102 (with an amino acid sequence represented by SEQ ID NO: 9) binds to the F70Cf's active site, it also displaces the C70f labelled-sidechain into the solvent and leads to a stronger fluorescence signal (right panel of FIG. 10B).

In order to confirm the molecular modeling results, a proteolytic study on F70Cf was performed. For performing the proteolytic study of F70Cf, the mass ratio of 20:1 between F70Cf and trypsin was used in 50 μM ZnCl₂, 20 mM, pH7.0, Tris-HCl buffer. The fluorescence spectra of the trypsinized samples were recorded by excitation at 494 nm at various time intervals (after 0, 1, 3, 9 and 18 h trypsinization) and recorded by Agilent Cary Eclipse Fluorescence Spectrophotometer (Agilent) (n=3 measurements) after exposing the trypsinized samples conjugated with fluorescein label to the buffer. The results are shown in FIG. 10C. After 1-18 h of trypsin digestion, digested fragments with the fluorescein label were exposed to the external aqueous environment, leading to an increase in fluorescence in a time-dependent manner. These findings are consistent with molecular modeling results.

### Example 3—Detection and Screening of Potential Drug Candidates for Inhibiting or Inactivating NDM-1 Enzyme Based on the Present Biosensor

For high-throughput inhibitor screening experiments, the fluorescence signal changes were recorded by adding 10 μL of each inhibitor (final concentration: 200 μM) automatically into 250 μL of F70Cf (final concentration: 0.13 μM) in a 96-well plate containing 50 μM ZnCl₂, 20 mM, pH7.0, Tris-HCl buffer, using Thermo Scientific Varioskan LUX Multimode Microplate Reader (Thermo Fisher Scientific) (n=3). The excitation and emission wavelengths were 494 and 520 nm, respectively. The injection of each inhibitor started at 0.5 min.

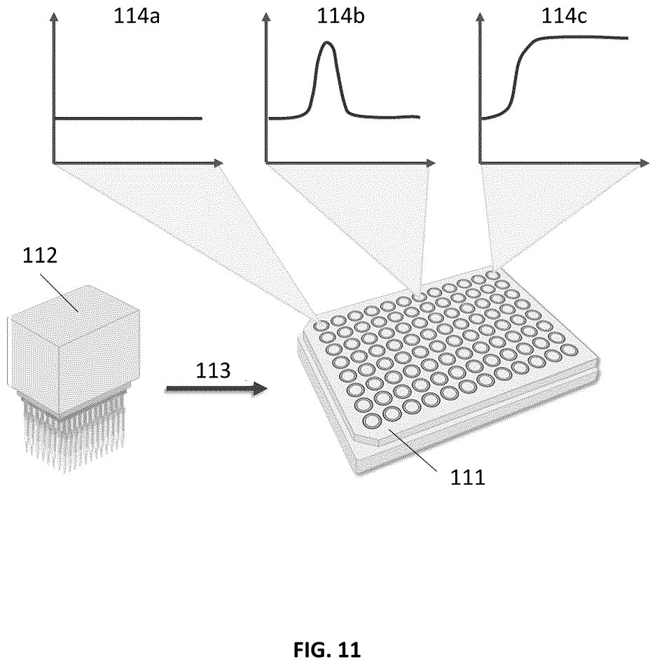

FIG. 11 schematically depicts how the present biosensor be used in high throughput screening of potential candidates of β-lactam antibiotics or β-lactamase inhibitors. Fluorescein labelled NDM-1 mutant, F70Cf, described in Example 1 or any other fluorophore labelled NDM-1 mutant according to certain embodiments of the present invention was immobilized in a microplate 111, e.g., a 96-well plate. A set of potential drug candidates from a corresponding compound library 112 was prepared before subjecting to the 96-well plate immobilized with the biosensor 113. Any fluorescence microplate reader can be used for sensing the fluorescence signals from the fluorophore labelled NDM-1 mutant or F70Cf. Three possible patterns of fluorescence signals at a function of time may be obtained: substantially no fluorescence signal (a plateau) (114a); a sharp increase in fluorescence intensity right after an injection of the potential candidate followed by a rapid decline (114b); a significant increase in fluorescence intensity until reaching a saturation and the high level of fluorescence intensity can be sustained over the time course (114c). In the time-course fluorescence signal pattern 114b, the potential candidate is likely a β-lactam antibiotic. In the time-course fluorescence signal pattern 114c, the potential candidate is likely a β-lactamase inhibitor, more specifically, an MBL inhibitor, or even more specifically, an NDM-1 inhibitor. The time-course fluorescence signal pattern 114a represents no binding activity occurs that will trigger “light on” of the fluorescein such as F5M conjugated specifically to a cysteine residue not in the active binding pocket. By this fast and easy preparation and measurement method, high-throughput screening of potential candidates of both β-lactam antibiotic and β-lactamase inhibitor can be in a one-pot detection platform. This platform is also applicable in evaluating clinical effectiveness of existing β-lactam antibiotic for infections arising from antibiotic-resistant bacteria or pathogens.

Although the invention has been described in terms of certain embodiments, other embodiments apparent to those of ordinary skill in the art are also within the scope of this invention. Accordingly, the scope of the invention is intended to be defined only by the claims which follow.

### INDUSTRIAL APPLICABILITY

The present biosensor is applicable in high-throughput drug screening of potential candidates of beta-lactams or beta-lactamase inhibitors, in particular, specific beta-lactams against MBL-containing Gram-negative bacteria or specific MBL inhibitors. The present invention is also a rapid, routine, and accurate tool to evaluate the clinical effectiveness of certain therapeutic regimes for treating infections arising from carbapenem-resistant pathogens.

What is claimed is:

1. A protein comprising a polypeptide having at least 95% sequence homology with an amino acid sequence selected from a group consisting of SEQ ID NOs: 1 to 5, wherein the amino acid at position 70 of SEQ ID NOs: 1-5 must be cysteine.

2. The protein of claim 1, wherein the polypeptide has at least 99% sequence homology with an amino acid sequence selected from a group consisting of SEQ ID NOs: 1 to 5.

3. The protein of claim 1, wherein the polypeptide has at least 99% sequence homology with SEQ ID NO: 1.

4. The protein of claim 1, wherein the polypeptide comprises SEQ ID NO: 1.

5. A biosensor comprising the protein according to claim 1 and a fluorophore, wherein the fluorophore is covalently conjugated via an optional linker to a side chain of the cysteine at position 70.

6. The biosensor of claim 5, wherein the polypeptide has at least 99% sequence homology with an amino acid sequence selected from a group consisting of SEQ ID NOs: 1 to 5.

7. The biosensor of claim 5, wherein the polypeptide has at least 99% sequence homology with SEQ ID NO: 1.

8. The biosensor of claim 5, wherein the polypeptide comprises SEQ ID NO: 1.

9. The biosensor of claim 5, wherein the polypeptide is SEQ ID NO: 6.

10. The biosensor of claim 5, wherein the fluorophore comprises a fluorescein or BADAN.

11. The biosensor according to any claim 5, wherein the fluorophore comprises a moiety selected from the group consisting of: 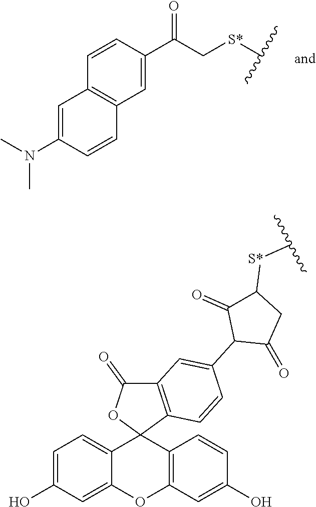
or a conjugate thereof, wherein S* represents the sulfur in the side chain of the cysteine at position 70.

12. A method for identifying a beta-lactam susceptible to hydrolysis by a New Delhi metallo-beta-lactamase, the method comprising contacting the beta-lactam with the biosensor according to claim 5 and detecting a change in fluorescence from the biosensor.

13. A method for identifying a beta-lactamase inhibitor, the method comprising contacting the biosensor according to claim 5 with a test compound and detecting a change in fluorescence from the biosensor.

14. A method for preparing the biosensor according to claim 5, the method comprising:
providing a protein comprising a polypeptide having at least 95% sequence homology with an amino acid sequence selected from a group consisting of SEQ ID NOs: 1 to 5, wherein the amino acid at position 70 of SEQ ID NOs: 1-5 must be cysteine; and
contacting the protein with a fluorescent labelling agent thereby forming the biosensor.

15. The method of claim 14, wherein the polypeptide has at least 99% sequence homology with an amino acid sequence selected from a group consisting of SEQ ID NOs: 1 to 5.

16. The method of claim 14, wherein the polypeptide has at least 99% sequence homology with SEQ ID NO: 1.

17. The method of claim 14, wherein the polypeptide comprises SEQ ID NO: 1.

18. The method according to claim 14, wherein the fluorescent labelling agent comprises a fluorescein or BADAN.

19. The method according to claim 14, wherein the fluorescent labelling agent is fluorescein-5-malemide (F5M).

20. The method of claim 19, wherein the protein and F5M are contacted in a molar ratio of 1:1-1.3, respectively.


SEQUENCE LISTING (9 sequences)

SEQ ID NO: 1 - protein, 270 aa, unidentified
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAVVTHAHQDKMGGMDALHAAGIATYANALSNQLAPQEGMVAAQHSLTFAANGWVEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 2 - protein, 270 aa, unidentified
MELPNIMHPVAKLSTALAAALMLSGCMAGEIRPTIGQQMETGDQRFGDLVFRQLAPNVWQHTSYLDMPGFGAVASNGLIVRDGGRVLVVDTAWTDDQTAQILNWIKQEINLPVALAVVTHAHQDKMGGMDALHAAGIATYANALSNQLAPQEGMVAAQHSLTFAANGWVEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 3 - protein, 270 aa, unidentified
MELPNIMHPVAKLSTALAAALMLSGCMAGEIRPTIGQQMETGDQRFGDLVFRQLAPNVWQHTSYLDMPGFGAVASNGLIVRDGGRVLVVDTAWTDDQTTQILNWIKQEINLPVALAVVTHAHQDKMGGMDALHAAGIATYANALSNQLALQEGMVAAQHSLTFAANGWVEPATAPNFGPLKVFYPGPGHTSDNITVGIDGTDIAFGGCLIKDSKAKSLGNPGDADTEHYAASARAFGAAFPKASMIVMSHSAPDSRAAITHTARMADKLR

SEQ ID NO: 4 - protein, 270 aa, unidentified
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAVVTHAHQDKMGGMDALHAAGIATYANALSNQLAPQEGLVAAQHSLTFAANGWVEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 5 - protein, 270 aa, unidentified
MELPNIMHPVAKLSTALAAALMLSGCMPGEIRPTIGQQMETGDQRFGDLVFRQLAPNVWQHTSYLDMPGFGAVASNGLIVRDGGRVLLVDTAWTDDQTAQILNWIKQEINLPVALAVVTHAHQDKMGGMDALHAAGIATYANALSNQLAPQEGLVAAQHSLTFAANGWVEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 6 - protein, 270 aa, synthetic construct
  SIGNAL 70: note = C70 labelled with fluorescein-5-malemide (F5M)
MELPNIMHPVAKLSTALAAALMLSGCMPGEIRPTIGQQMETGDQRFGDLVFRQLAPNVWQHTSYLDMPGCGAVASNGLIVRDGGRVLVVDTAWTDDQTAQILNWIKQEINLPVALAVVTHAHQDKMGGMDALHAAGIATYANALSNQLAPQEGMVAAQHSLTFAANGWVEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 7 - DNA, 30 bp, synthetic construct
  source 1..30: mol_type = other DNA
gacatgccgggttgcggggcagtcgcttcc

SEQ ID NO: 8 - DNA, 30 bp, synthetic construct
  source 1..30: mol_type = other DNA
ggaagcgactgccccgcaacccggcatgtc

SEQ ID NO: 9 - protein, 21 aa, Podisus maculiventris
GSKKPVPIIYCNRRTGKCQRM